(19) United States
(12) Patent Application Publication
(10) Pub. No.: US 2024/0324847 A1
(43) Pub. Date: Oct. 3, 2024

(54) ENDOSCOPE SYSTEM

(71) Applicant: OLYMPUS CORPORATION, Tokyo (JP)

(72) Inventors: Hisatsugu TAJIMA, Tokyo (JP); Ryota SASAI, Tokyo (JP); Noriaki YAMANAKA, Tokyo (JP)

(73) Assignee: OLYMPUS CORPORATION, Tokyo (JP)

(21) Appl. No.: 18/738,750

(22) Filed: Jun. 10, 2024

Related U.S. Application Data
(63) Continuation of application No. PCT/JP2021/048541, filed on Dec. 27, 2021.

(51) Int. Cl. A61B 1/00

(52) U.S. Cl. CPC A61B 1/00006; A61B 1/00009; A61B 1/0016

(57) ABSTRACT

An endoscope system includes an endoscope, a robot arm that moves the endoscope, and a controller that controls the endoscope and the robot arm. The controller is configured to receive an image acquired by the endoscope, calculate a first orientation of a first treatment tool in a first image, which is the image received at a given point in time, calculate a second orientation of a second treatment tool in a second image, which is the image received a predetermined time before the first image, calculate a difference between the first orientation and the second orientation, determine whether or not the endoscope follows movement of the first treatment tool by comparing the difference with a threshold value, and in response to determining that the endoscope follows the movement of the first treatment tool, generate a command for the robot arm for making the endoscope follow the first treatment tool.

### CROSS-REFERENCE TO RELATED APPLICATIONS

This is a continuation of International Application PCT/JP2021/048541, with an international filing date of Dec. 27, 2021, which is hereby incorporated by reference herein in its entirety.

### TECHNICAL FIELD

The present disclosure relates to an endoscope system, and particularly relates to an endoscope system in which an endoscope is made to follow a follow-target treatment tool.

### BACKGROUND

In the related art, there is a known endoscope system in which an endoscope is made to follow a treatment tool of interest, such as a treatment tool held in the right hand of an operator or a marked treatment tool (for example, see PTL 1).

### CITATION LIST

### Patent Literature

PTL 1 U.S. Pat. No. 10,028,792 Specification

### SUMMARY

An aspect of the present disclosure is directed to an endoscope system comprising: an endoscope; a robot arm that is configured to move the endoscope; and a controller that is configured to control the endoscope and the robot arm, wherein the controller is configured to: receive an image acquired by the endoscope, calculate a first orientation of a first treatment tool in a first image, which is the image received at a given point in time, calculate a second orientation of a second treatment tool in a second image, which is the image received a predetermined time before the first image, calculate a difference in orientation between the first orientation and the second orientation, determine whether or not the endoscope follows movement of the first treatment tool by comparing the difference in orientation with a threshold value, and in response to determining that the endoscope follows the movement of the first treatment tool, generate a command for the robot arm for making the endoscope follow the first treatment tool.

### BRIEF DESCRIPTION OF DRAWINGS

FIG. 1A is an external view of the overall configuration of an endoscope system according to one embodiment of the present disclosure.

FIG. 1B is a view for explaining the movements of an endoscope and a treatment tool inserted into a body via ports in a body wall.

FIG. 2A is a block diagram showing the hardware configuration of a control device of the endoscope system shown in FIG. 1.

FIG. 2B is a block diagram showing functions of the control device of the endoscope system shown in FIG. 1.

FIG. 3A shows an example endoscopic image acquired during surgery.

FIG. 3B shows another example endoscopic image acquired during the surgery.

FIG. 4A is a view for explaining an example of a follow-target setting method.

FIG. 4B is a view for explaining another example of a follow-target setting method.

FIG. 5A is a view for explaining an example of a treatment-tool recognition method.

FIG. 5B is a view for explaining another example of a treatment-tool recognition method.

FIG. 6 is a view for explaining examples of orientations of treatment tools.

FIG. 7 is a flowchart of a follow method according to a first embodiment.

FIG. 8A is a flowchart of a modification of the follow method according to the first embodiment.

FIG. 8B is a flowchart of a candidate narrowing-down routine in the FIG. 8A.

FIG. 9 shows example endoscopic images during replacement of a follow-target treatment tool.

FIG. 10 is a flowchart of a follow method according to a second embodiment.

FIG. 11 is a view for explaining an example of the position of a treatment tool.

FIG. 12A is a view for explaining a follow-target determination method based on pivot points.

FIG. 12B is a view for explaining the follow-target determination method based on pivot points.

FIG. 13 is a flowchart of a follow method according to a third embodiment.

FIG. 14A is a flowchart of a modification of the follow method according to the third embodiment.

FIG. 14B is a flowchart of a candidate narrowing-down routine in FIG. 14A.

FIG. 15 is a flowchart of a modification of the follow method according to the third embodiment.

FIG. 16 is a view for explaining a method for estimating a pivot point from an endoscopic image.

FIG. 17 is a flowchart of a follow method according to a fourth embodiment.

FIG. 18 is a view showing examples of the orientation and the position of a treatment tool, which are superimposed on an endoscopic image.

### DESCRIPTION OF EMBODIMENTS

### First Embodiment

An endoscope system according to a first embodiment of the present disclosure will be described below with reference to the drawings.

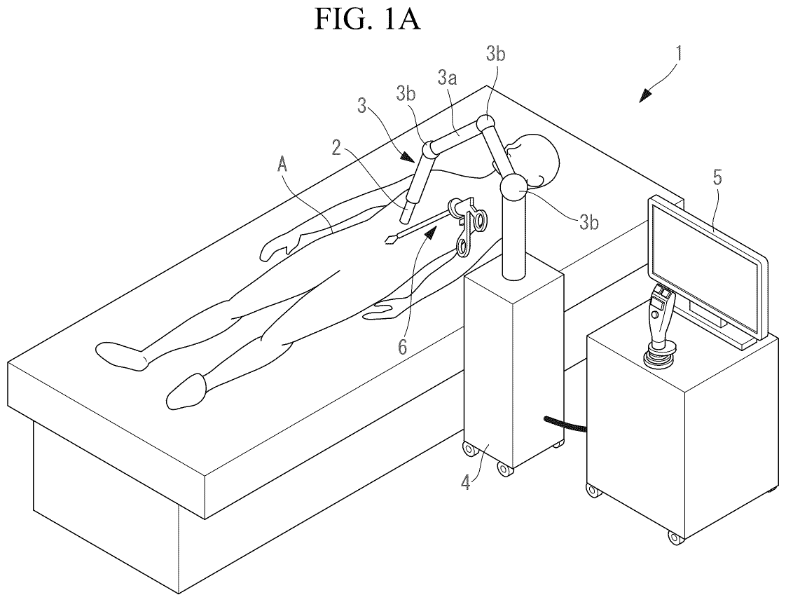

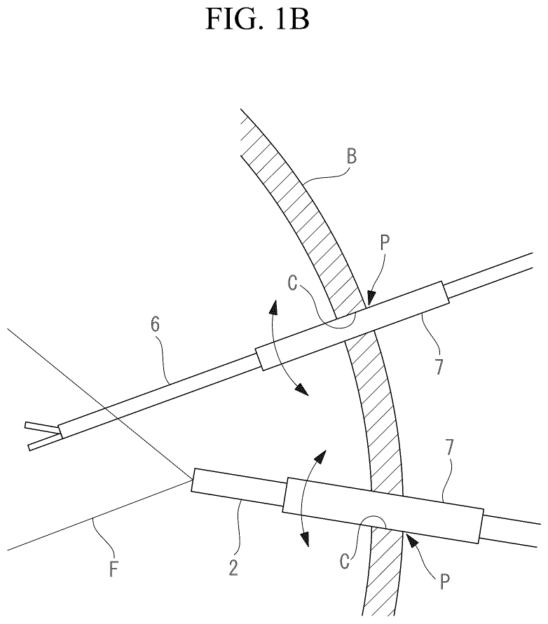

As shown in FIGS. 1A and 1B, an endoscope system 1 of this embodiment is used for surgery in which an endoscope 2 and one or more treatment tools 6 are used, as in laparoscopic surgery, for example. Although only one treatment tool 6 is used in FIGS. 1A and 1B, two or more treatment tools 6 may be used.

As shown in FIG. 1B, a plurality of holes C are formed in a body wall B of a patient A as ports through which the endoscope 2 and the one or more treatment tools 6 are inserted into the body. Each of the endoscope 2 and the one or more treatment tools 6 is inserted into the body via a trocar 7 inserted in any of the ports C and is supported by the body wall B so as to be pivotable about a predetermined pivot point P that corresponds to the position of the port C, whereby the position and the orientation of each of the endoscope 2 and the one or more treatment tools 6 in the body can be changed through a pivotal movement about the pivot point P.

The endoscope system 1 includes: the endoscope 2; a movement device 3 that holds the endoscope 2 to move the endoscope 2 in the body; a control device 4 that is connected to the endoscope 2 and the movement device 3 to control the endoscope 2 and the movement device 3; and a display device 5 that displays an endoscopic image.

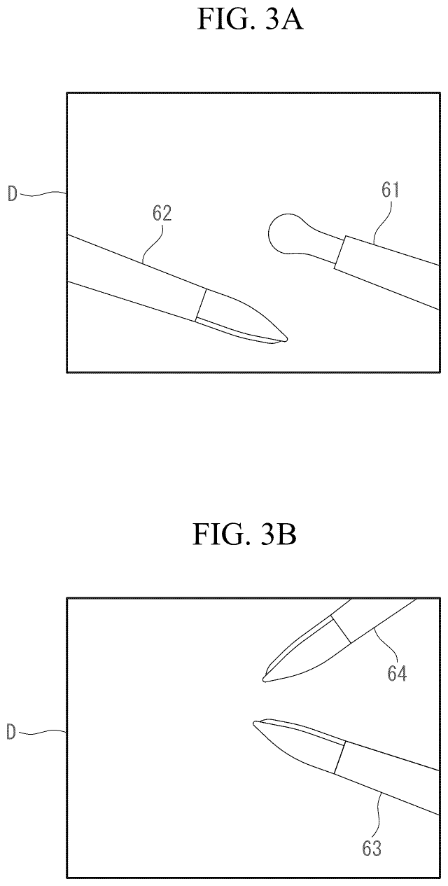

The endoscope 2 is a rigid scope, for example, and includes an image acquisition unit (not shown) that has an image acquisition element and that acquires an endoscopic image. As shown in FIGS. 3A and 3B, the endoscope 2 acquires endoscopic images D including one or more treatment tools 61, 62, 63, and 64 by means of the image acquisition unit and sends the endoscopic images D to the control device 4.

The movement device 3 includes a robot arm 3a having a plurality of joints 3b and holds a proximal-end section of the endoscope 2 at a distal-end section of the robot arm 3a. The movement device 3 can move the endoscope 2 due to the movements of the joints 3b to change the position and the orientation of the endoscope 2.

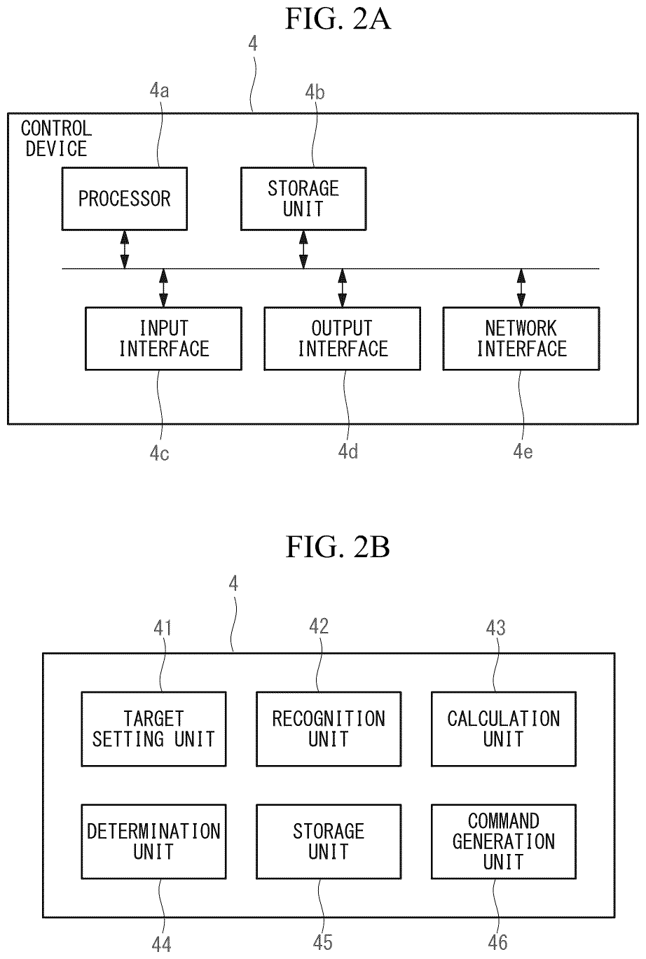

As shown in FIG. 2A, the control device 4 includes at least one processor 4a such as a central processing unit, a storage unit 4b, an input interface 4c, an output interface 4d, and a network interface 4e.

The endoscopic images D sent from the endoscope 2 are sequentially input to the control device 4 via the input interface 4c, are sequentially output to the display device 5 via the output interface 4d, and are displayed on the display device 5. While observing the endoscopic images D displayed on the display device 5, an operator operates the treatment tools 61, 62, 63, and 64, which are inserted into the body, to perform treatment of an affected area in the body by means of the treatment tools 61, 62, 63, and 64. The display device 5 is an arbitrary display such as a liquid crystal display.

The storage unit 4b includes a volatile working memory, such as a RAM (random access memory), and a non-volatile recording medium, such as a ROM (read-only memory) or a hard disk, and the non-volatile recording medium stores a program and data necessary to cause the processor 4a to execute processing. Functions of the control device 4, to be described layer, are realized when the program is executed by the processor 4a. Some of the functions of the control device 4 may be realized by a specialized logic circuit or the like.

As shown in FIGS. 3A and 3B, the kinds, the arrangement, and the number of treatment tools in the endoscopic images D are changed during surgery. For example, the energy device 61 is changed to the forceps 63, and, after the treatment tool 62 is removed, the other treatment tool 64 is inserted via another port.

The control device 4 sets the treatment tool 61, which is one of the treatment tools, in the endoscopic image D to a follow target and controls the movement device 3 on the basis of the position of the follow target 61, thereby making the endoscope 2 follow the follow target 61 to keep capturing the follow target 61 in a field F of view of the endoscope 2. For example, the control device 4 controls the movement device 3 on the basis of the position of the follow target 61 in the endoscopic image D such that the distal end of the follow-target treatment tool 61 is arranged in a predetermined central area in the endoscopic image D.

Specifically, as shown in FIG. 2B, the control device 4 includes: a target setting unit 41 that sets the treatment tool 61 to the follow target; a recognition unit 42 that recognizes the treatment tools 61 and 62 present in the current endoscopic image D; a calculation unit 43 that calculates the current orientations of the treatment tools 61 and 62 present in the current endoscopic image D; a determination unit 44 that determines whether or not the endoscope 2 follows the movements of the treatment tools 61 and 62, on the basis of the current orientations of the treatment tools 61 and 62 and a past orientation of the follow target 61; a storage unit 45 that stores the current orientation of the follow-target treatment tool 61 when it is determined that the endoscope 2 follows the treatment tool 61; and a command generation unit 46 that generates a command for the movement device 3.

The target setting unit 41 sets the treatment tool 61, which is one of the treatment tools, to a follow target automatically or sets the treatment tool 61, which is one of the treatment tools, to a follow target on the basis of an operation of the operator.

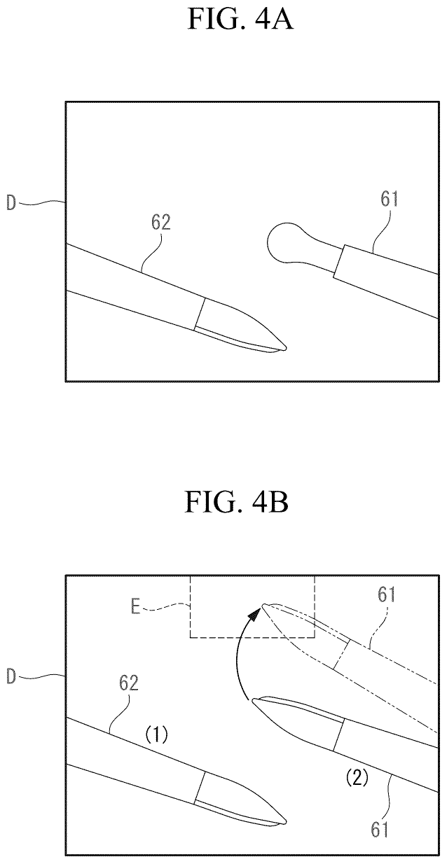

In the case where the treatment tool 61 is set to a follow target automatically, as shown in FIG. 4A, the treatment tool that is present in the endoscopic image D and that satisfies a predetermined condition is set to a follow target.

In one example, the predetermined condition is the kind of the treatment tool. For example, an energy device is a treatment tool to which a doctor pays attention during treatment, and only one energy device is usually present in the endoscopic image D. The target setting unit 41 recognizes the kinds of the treatment tools 61 and 62 in the endoscopic image D and sets the energy device 61, such as an electric scalpel, to a follow target.

In the case where the treatment tool 61 is set to a follow target manually, as shown in FIG. 4B, the desired treatment tool 61 is moved to a predetermined cursor E set in the endoscopic image D, whereby the desired treatment tool 61 is set to a follow target. Alternatively, a number assigned to each of the treatment tools 61 and 62 or a number assigned to each of the ports is specified by the operator, whereby the desired treatment tool 61 is set to a follow target.

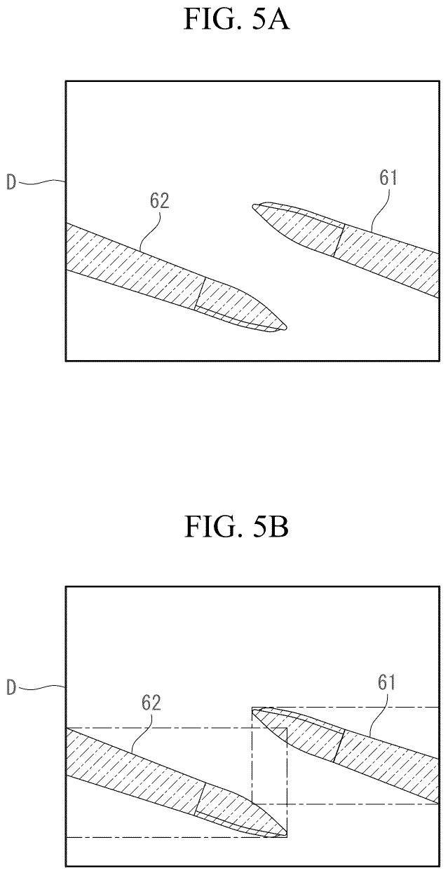

The recognition unit 42 applies processing to the current endoscopic image D, which is the latest endoscopic image acquired by the endoscope 2, thereby recognizing all the treatment tools 61 and 62 present in the current endoscopic image D. In order to recognize the treatment tools 61 and 62, a known image recognition technology using deep learning, such semantic segmentation shown in FIG. 5A or instance segmentation shown in FIG. 5B, is used.

The calculation unit 43 calculates 2D or 3D current orientations of all the treatment tools 61 and 62 recognized by the recognition unit 42.

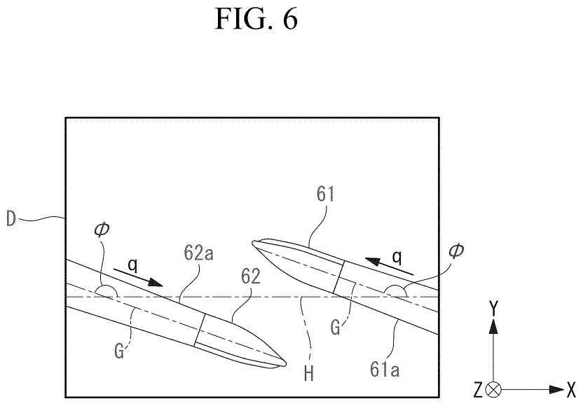

As shown in FIG. 6, examples of 2D orientations of the treatment tools 61 and 62 are vectors q in the longitudinal directions of the treatment tools 61 and 62 on an image plane (XY plane) of the endoscopic image D or angles φ formed by a predetermined reference axis H in the endoscopic image D and longitudinal axes G of the treatment tools 61 and 62.

Examples of 3D orientations of the treatment tools 61 and 62 are vectors q in the longitudinal directions of the treatment tools 61 and 62 in a 3D space of the endoscopic image D. Other examples of 3D orientations of the treatment tools 61 and 62 are angles φ of the treatment tools 61 and 62 on the image plane, tilt angles of the treatment tools 61 and 62 in the depth direction (Z-direction) perpendicular to the image plane, and angles formed by the X axis or the Y axis (the horizontal axis or the vertical axis of the endoscopic image D) and the longitudinal axes G. The vectors q or the angles are calculated from an endoscopic image D that is a stereo image including information of the 3D positions of the treatment tools 61 and 62, or are obtained by an optical or magnetic attitude sensor. By using the fact that the thicknesses of shafts 61a and 62a of the treatment tools 61 and 62 in the endoscopic image D change along the depth direction, the vectors or the angles may be calculated from the thicknesses of the shafts 61a and 62a in a 2D endoscopic image D.

The determination unit 44 obtains a past orientation (second orientation) of the follow target (second treatment tool) from the storage unit 45. The past orientation is the orientation of the follow target in a past endoscopic image D, which is the orientation stored in the storage unit 45 a predetermined time before the current orientation, as will be described later.

Next, the determination unit 44 determines whether or not the endoscope 2 follows the movements of the treatment tools 61 and 62, on the basis of the current orientations of the treatment tools 61 and 62 and the past orientation of the follow target.

Specifically, the determination unit 44 compares the current orientation (first orientation) of the treatment tool (first treatment tool) 61 with the past orientation of the follow target. The determination unit 44 determines that the endoscope 2 follows the movement of the treatment tool 61, in the case where the current orientation of the treatment tool 61 is the same or substantially the same as the past orientation of the follow target, and determines that the endoscope 2 does not follow the movement of the treatment tool 61, in the case where the current orientation of the treatment tool 61 is neither the same nor substantially the same as the past orientation of the follow target.

For example, the determination unit 44 calculates a deviation d that is the amount of deviation of the current orientation of the treatment tool 61 from the past orientation of the follow target. The determination unit 44 determines that the current orientation of the treatment tool 61 is the same or substantially the same as the past orientation of the follow target, in the case where the magnitude of the deviation d is equal to or smaller than a predetermined threshold, and determines that the current orientation of the treatment tool 61 is neither the same nor substantially the same as the past orientation of the follow target, in the case where the magnitude of the deviation d is larger than the predetermined threshold.

Similarly, on the basis of whether or not the current orientation (third orientation) of the other treatment tool (third treatment tool) 62 is the same or substantially the same as the past orientation of the follow target, the determination unit 44 determines whether or not the endoscope 2 follows the movement of the other treatment tool 62.

The treatment tool of which the current orientation is the same or substantially the same as the past orientation of the follow target is a candidate for the follow target. In the case where the current orientation of only the treatment tool 61 is the same or substantially the same as the past orientation of the follow target, i.e., in the case where only one candidate is present, the determination unit 44 determines that this candidate treatment tool 61 is the follow target and determines that the endoscope 2 follows the movement of the treatment tool 61. On the other hand, in the case where the current orientations of the plurality of treatment tools 61 and 62 are the same or substantially the same as the past orientation of the follow target, i.e., in the case where a plurality of candidates are present, the determination unit 44 determines that, of the plurality of candidates 61 and 62, the treatment tool 61, of which the magnitude of the deviation d is the smallest, is the follow target and finally determines that the endoscope 2 follows the movement of the treatment tool 61.

Since the plurality of treatment tools 61 and 62 are inserted into the body from the ports C, which are different from each other, the orientations of the plurality of treatment tools 61 and 62 are different from each other, and the orientations of the treatment tools 61 and 62 can be changed within limited ranges. That is, the orientation of the follow-target treatment tool 61 is different from the orientation of the other treatment tool 62 and is constant or substantially constant so long as the position and the orientation of the endoscope 2 are not changed. Therefore, it is possible to identify that the treatment tool 61, the current orientation of which is the same or substantially the same as the past orientation, is the follow target.

In the case where it is determined that the endoscope 2 does not follow the movements of all the treatment tools 61 and 62, e.g., in the case where the deviations d of all the treatment tools 61 and 62 are larger than the threshold, the determination unit 44 determines that the treatment tool corresponding to the follow target is not present in the endoscopic image D and notifies the determination result to the target setting unit 41. In response to the notification from the determination unit 44, the target setting unit 41 sets a follow target again.

In one example, the deviation d is calculated from Equation (1), where q^{t} is a vector representing the current orientation, and q^{t-i} is a vector representing the past orientation of the follow target. In Equation (1), the individual terms may be weighted.

$$d = ‖ {q}^{t} - {q}^{t - i} ‖ \qquad ( 1 )$$

In another example, the deviation of the orientation may be the angle θ formed by the orientation vectors q^{t} and q^{t-i}, as indicated by the following equation.

$$θ = {cos}^{- 1} ( {q}^{t} \cdot {q}^{t - i} )$$

It is possible that the recognition unit 42 also recognizes the kinds of the treatment tools 61 and 62, and the determination unit 44 determines the follow target on the basis of the kinds of the treatment tools 61 and 62 in addition to the current orientations thereof. In this case, the kind of the follow target may also be stored in the storage unit 45.

The storage unit 45 is formed of the storage unit 4b. The storage unit 45 stores the orientation of the follow target when the follow target is set by the target setting unit 41. This orientation is calculated by the calculation unit 43 from, for example, an endoscopic image D when the follow target is set. Thereafter, the storage unit 45 stores, among the current orientations calculated by the calculation unit 43, the current orientation of the treatment tool 61, which is determined to be the follow target by the determination unit 44. The orientation that has already been stored is replaced with the newly calculated current orientation, whereby the orientation stored in the storage unit 45 is updated. The current orientation stored in the storage unit 45 is used, as the past orientation, in determining the follow target to be executed thereafter by the determination unit 44.

The command generation unit 46 generates a command for the movement device 3 on the basis of the determination result of the determination unit 44 and sends the command to the movement device 3.

Specifically, in the case where it is determined that the endoscope 2 follows the movement of the treatment tool 61, the command generation unit 46 generates a command for making the endoscope 2 follow the treatment tool 61. On the other hand, in the case where it is determined that the endoscope 2 does not follow the movement of the treatment tool 61, the command generation unit 46 generates a command for making the endoscope 2 stop following the treatment tool 61.

Next, the operation of the endoscope system 1 will be described below.

The endoscope 2 and the one or more treatment tools 61 and 62 are inserted into the body via the trocars 7 in the different ports C, and endoscopic images D including the one or more treatment tools 61 and 62 are acquired by the endoscope 2. The operator performs treatment by using the one or more treatment tools 61 and 62 while observing the endoscopic images D displayed on the display device 5.

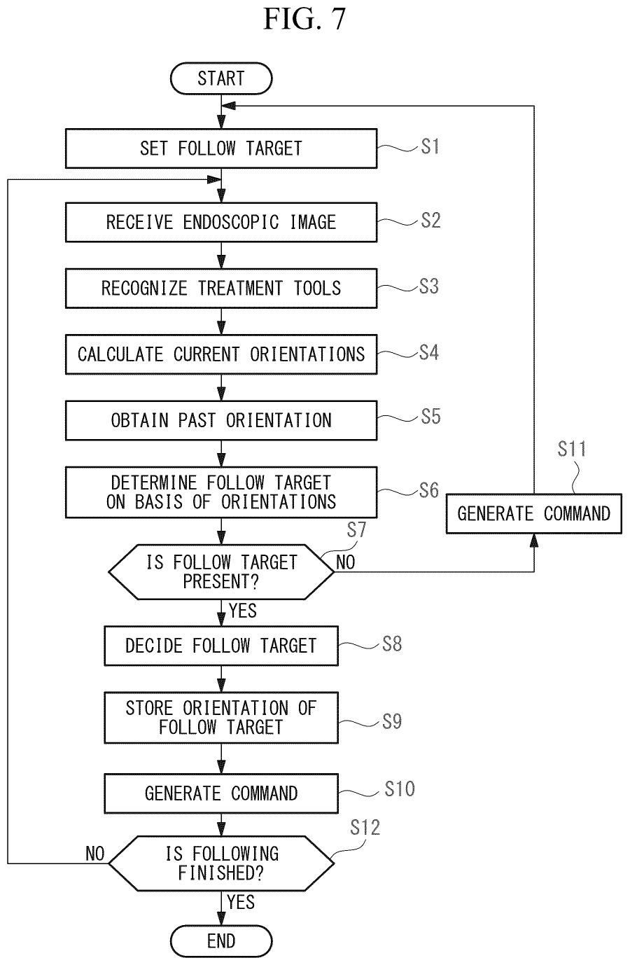

During the treatment with the treatment tools 61 and 62, the control device 4 executes a follow method for making the endoscope 2 follow the follow-target treatment tool 61, which is moved in the body, as shown in FIG. 7.

The follow method includes: Step S1 for setting the treatment tool 61 to a follow target; Steps S2 to S4 for calculating the current orientations of the treatment tools 61 and 62 in the endoscopic image D; Steps S5 to S8 for determining the follow target on the basis of the current orientations; Step S9 for storing the current orientation of the follow target; and Steps S10 and S11 for generating a command for the movement device 3 on the basis of the determination result of the follow target.

First, the target setting unit 41 sets the treatment tool 61, which is one of the treatment tools, in the endoscopic image D to a follow target (Step S1). After the setting of the follow target, the control device 4 starts to control the movement device 3, whereby the endoscope 2 starts to follow the follow target 61. For example, the control device 4 calculates the 2D or 3D position of the follow target 61 on the basis of the endoscopic image D and makes the movement device 3 move the endoscope 2 such that the follow target 61 is arranged in the central area of the endoscopic image D.

The control device 4 executes processing of S2 to S11 for determining and tracking the follow target 61 in parallel with the control of the movement device 3.

First, the recognition unit 42 receives the current endoscopic image D from the endoscope 2 (Step S2) and recognizes all the treatment tools 61 and 62 in the current endoscopic image D (Step S3). Next, the calculation unit 43 calculates the current orientations of all the treatment tools 61 and 62 in the current endoscopic image D (Step S4).

Next, the determination unit 44 obtains a past orientation of the follow target 61 from the storage unit 45 (Step S5). The past orientation obtained at this time is the orientation stored in the storage unit 45 in Step S9 in the previous loop, which is the orientation the predetermined time before the current orientation.

Then, the determination unit 44 determines whether or not the endoscope 2 follows the movements of the treatment tools 61 and 62, i.e., whether or not the treatment tools 61 and 62 are the follow targets, on the basis of the current orientations of the treatment tools 61 and 62 and the past orientation of the follow target 61 (Step S6). For example, the determination unit 44 calculates deviations d of the current orientations of the treatment tools 61 and 62 from the past orientation and determines whether or not each of the deviations d of the treatment tools 61 and 62 is equal to or smaller than the predetermined threshold and is the smallest.

In the case where the follow-target treatment tool 61 determined to be followed by the endoscope 2 is present (YES in Step S7), the determination unit 44 decides that the treatment tool 61 is the follow target (Step S8). Next, the storage unit 45 stores the current orientation of the treatment tool 61 decided as being the follow target (Step S9). Furthermore, the command generation unit 46 generates a command for making the endoscope 2 follow the treatment tool 61 (Step S10) and sends the command to the movement device 3.

On the other hand, in the case where the treatment tool corresponding to the follow target is not present (NO in Step S7), the command generation unit 46 generates a command for making the endoscope 2 stop following the treatment tool 61 (Step S11) and sends the command to the movement device 3. Thereafter, the target setting unit 41 again sets one treatment tool in the endoscopic image D to a follow target (Step S1), and Steps S2 to S11 are executed again.

Steps S1 to S11 are repeated until following is finished in Step S12.

In this way, according to this embodiment, the current orientations of the treatment tools 61 and 62 in the current endoscopic image D are calculated, and whether or not the treatment tools 61 and 62 are the follow targets is determined on the basis of the current orientations. Since the orientations of the treatment tools 61 and 62 are limited by the ports C and the trocars 7, the orientation of the follow-target treatment tool 61 in the endoscopic image D is not usually rapidly changed. Furthermore, the orientations of the plurality of treatment tools 61 and 62 in the endoscopic image D are different from each other. Therefore, it is possible to accurately determine whether or not the treatment tools 61 and 62 are the follow targets, on the basis of the current orientations of the treatment tools 61 and 62.

Furthermore, a deviation d of the current orientation with respect to the past orientation of the follow target is calculated, and the follow target is determined on the basis of the deviation d. Accordingly, the follow target can be determined merely by a simple calculation.

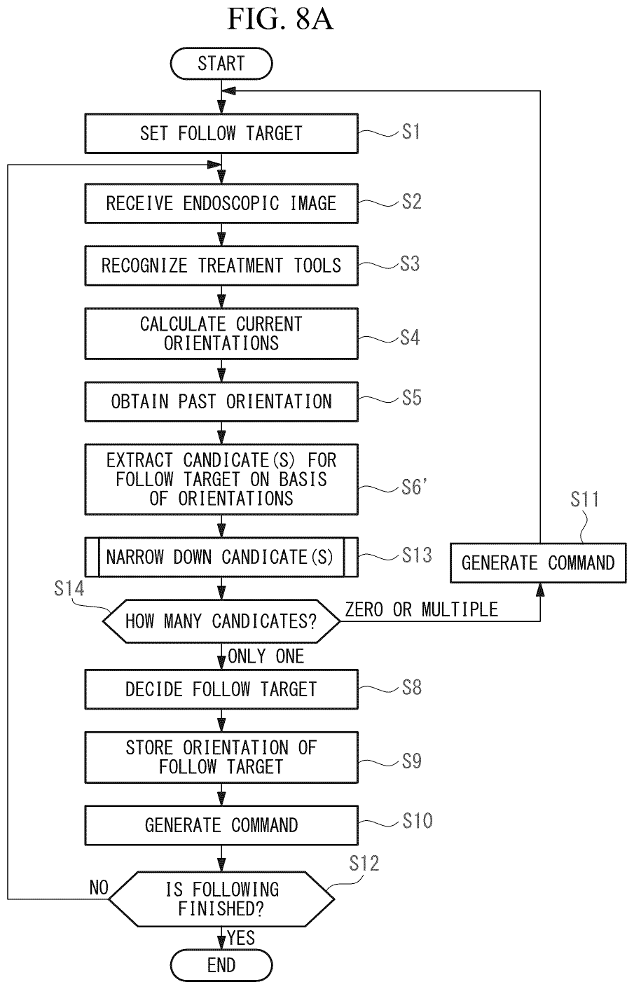

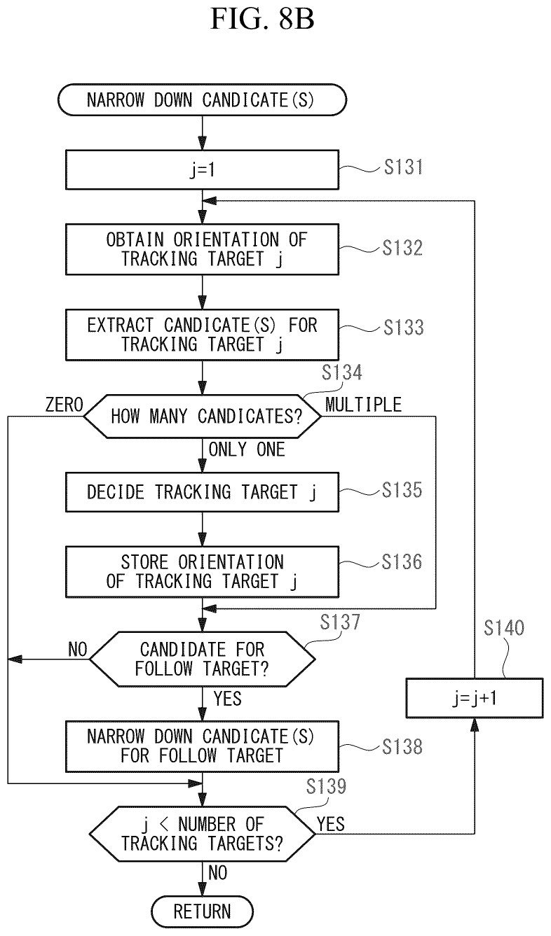

In this embodiment, although the determination unit 44 tracks and determines only the follow target on the basis of the orientation thereof, it is also possible to track and determine the other treatment tool as well on the basis of the orientation thereof, as shown in FIGS. 8A and 8B.

That is, in the case where the plurality of treatment tools 61 and 62 are included in the endoscopic image D, the target setting unit 41 sets the treatment tool 61, which is one of the treatment tools, to a follow target and sets the other treatment tool 62 to a tracking target, in Step S1. The determination unit 44 extracts a candidate(s) for the follow target from the plurality of treatment tools 61 and 62, on the basis of the current orientations of the treatment tools 61 and 62 and the past orientation of the follow target, for example, on the basis of the deviations d, in Step S6′. Then, the determination unit 44 narrows down the candidate(s) for the follow target to one by tracking the tracking target 62 (Step S13).

Specifically, as shown in FIG. 8B, the determination unit 44 selects one tracking target j (Step S131) and obtains the past orientation of the tracking target j from the storage unit 45 (Step S132). Next, similarly to Steps S6 and S6′, the determination unit 44 calculates a deviation d of the current orientation of the tracking target j from the past orientation and extracts, as a candidate for the tracking target j, the treatment tool of which the deviation d is equal to or smaller than the predetermined threshold (Step S133).

In the case where the number of candidates is only one (“only one” in Step S134), the determination unit 44 determines that candidate to be the tracking target j (Step S135) and updates the past orientation of the tracking target j stored in the storage unit 45 to the current orientation (Step S136). Next, in the case where the number of candidates is one or multiple (“only one” or “multiple” in Step S134), the determination unit 44 determines whether or not the tracking target j is included in the candidate(s) for the follow target (Step S137). In the case where the tracking target j is included in the candidate(s) for the follow target, the determination unit 44 deletes the tracking target j from the candidate(s) for the follow target (Step S138).

Thereafter, the determination unit 44 selects another tracking target j+1 (Step S140) and repeats Steps S132 to S138, thereby executing processing of Steps S132 to S138 for all tracking targets (Step S139). Accordingly, the candidate(s) for the follow target is narrowed down.

As shown in FIG. 8A, in the case where, after the candidate(s) is narrowed down in Step S13, the number of remaining candidates for the follow target is only one (Step S14), the determination unit 44 determines the remaining candidate to be the follow target (Step S8).

On the other hand, in the case where the number of remaining candidates is zero or multiple (Step S14), the determination unit 44 determines that the follow target is not present or that the follow target cannot be determined, and the target setting unit 41 sets a follow target again (Step S1).

In this way, according to this modification, all the treatment tools 61 and 62 included in the endoscopic image D are tracked, and the treatment tool that is likely to be the other treatment tool 62 is deleted from the candidate(s) for the follow target, thereby making it possible to narrow down the candidate(s) for the follow target. Accordingly, it is possible to suppress misrecognition of the follow target and to further improve the recognition accuracy of the follow target.

If the follow target is misrecognized, the endoscope 2 keeps following the wrong treatment tool thereafter. Therefore, it is important to prevent misrecognition of the follow target. In the case where the number of candidates for the follow target becomes zero or multiple as a result of narrowing down the candidate(s), a follow target is set again, thereby making it possible to prevent the endoscope 2 from keeping following the wrong treatment tool.

In the above-described embodiment, although, in the case where it is determined that the endoscope 2 follows the movements of the plurality of treatment tools 61 and 62, the determination unit 44 determines the follow target on the basis of the deviations d, and the command generation unit 46 generates a command for making the endoscope 2 follow the follow target, instead of this, it is also possible that the command generation unit 46 generates a command for making the endoscope 2 stop following all the treatment tools 61 and 62.

In this case, after whether or not the endoscope 2 follows the movements of the treatment tools (the first treatment tool and the third treatment tool) 61 and 62 is determined on the basis of the current orientations (the first orientation and the third orientation) of the treatment tools 61 and 62, the determination unit 44 generates a first result by determining whether or not the endoscope 2 follows the movement of the treatment tool 61 and generates a second result by determining whether or not the endoscope 2 follows the movement of the treatment tool (the third treatment tool) 62.

In the case where the first result is affirmative, and the second result is negative, the command generation unit 46 generates a command for making the endoscope 2 follow the treatment tool 61. Furthermore, in the case where the first result is negative, and the second result is affirmative, the command generation unit 46 generates a command for making the endoscope 2 follow the treatment tool 62. In this way, in the case where the number of candidates for the follow target is only one, the control device 4 controls the movement device 3 to make the endoscope 2 follow the follow target.

In the case where the first result and the second result are both affirmative, or in the case where the first result and the second result are both negative, the command generation unit 46 generates a command for making the endoscope 2 stop following the treatment tools 61 and 62. In this way, in the case where a plurality of candidates for the follow target are present or in the case where no candidate is present, the control device 4 controls the movement device 3 to make the endoscope 2 stop following the follow target.

In the above-described embodiment, after determining that the follow target is not present, the control device 4 may automatically place the follow target.

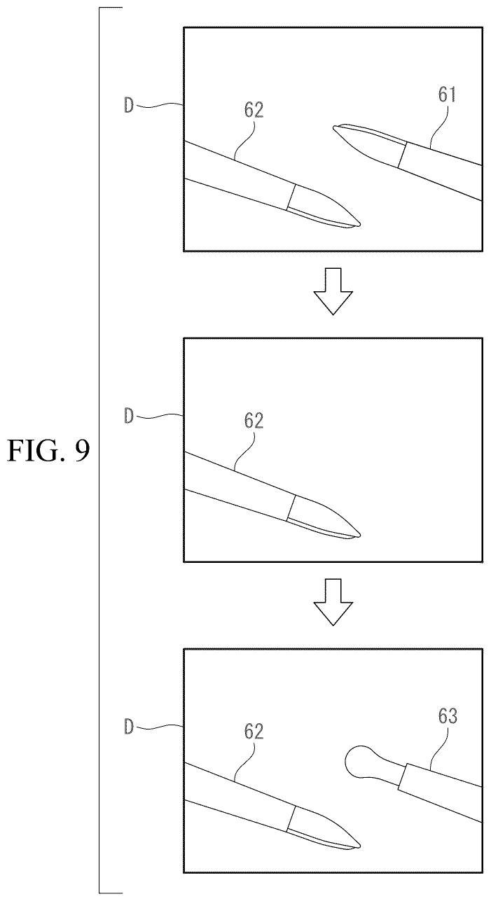

For example, in the case where the follow-target treatment tool 61 disappears from the endoscopic image D due to a replacement of the treatment tool or the like, as shown in FIG. 9, or in the case where a deviation d of the orientation of the follow-target treatment tool 61 temporarily exceeds the threshold for some reason, it is determined that the treatment tool corresponding to the follow target is not present, in Step S7, and the control device 4 loses sight of the follow target. In this case, after Step S7, the control device 4 executes again Steps S2 to S7 using the next endoscopic image D. The control device 4 repeats Steps S2 to S7 until the treatment tool 63, of which the deviation d is equal to or smaller than the threshold, is found, and sets the treatment tool 63, of which the deviation d is equal to or smaller than the threshold, to the follow target, thereby placing the follow target. Accordingly, the treatment tool 63, of which the current orientation is the same or substantially the same as the orientation of the follow target 61 immediately before it is determined that the follow target is not present, is automatically placed as the follow target.

For example, when the follow-target treatment tool 61 is replaced, the treatment tool 61 is removed from the body, whereby the follow target disappears from the endoscopic image D, and then, the other treatment tool 63 is inserted into the body, whereby the follow target appears again in the endoscopic image D. The orientation of the follow target does not change between before and after the follow target disappears, unless the endoscope 2 is not moved while the treatment tool 61 is replaced with the treatment tool 63. Therefore, even after the control device 4 once lost sight of the follow target, it is possible to recognize the follow target again on the basis of the current orientations of the treatment tools. Accordingly, it is possible to save the trouble of having to set the follow target again, at the time of treatment-tool replacement or the like.

In the above-described embodiment, although the storage unit 45 updates the orientation of the follow target every time the follow target is decided, instead of this, the storage unit 45 may add the current orientation to the past orientation, thereby storing the orientations of the follow target calculated at a plurality of points in time.

In this case, the determination unit 44 may use, in determination of the follow target, a plurality of past orientations of the follow target calculated at a plurality of past points in time. For example, the determination unit 44 may calculate deviations d of the current orientations with respect to each of the plurality of individual past orientations and determine the follow target on the basis of the plurality of deviations d.

### Second Embodiment

Next, an endoscope system according to a second embodiment of the present disclosure will be described below.

An endoscope system 1 of this embodiment differs from the first embodiment in that the follow target is determined on the basis of the current positions of the treatment tools 61 and 62 in addition to the current orientations thereof. In this embodiment, configurations different from those in the first embodiment will be described below, identical reference signs are assigned to configurations common to those in the first embodiment, and a description thereof will be omitted. As in the first embodiment, the endoscope system 1 of this embodiment includes the endoscope 2, the movement device 3, the control device 4, and the display device 5.

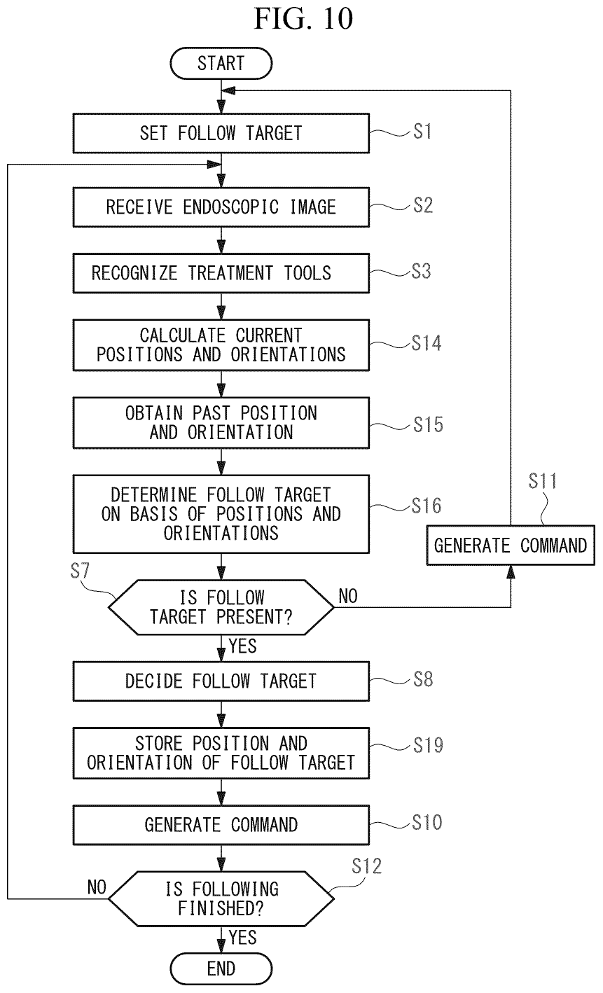

As shown in FIG. 10, the calculation unit 43 calculates, in addition to the current orientations, the current positions of the treatment tools 61 and 62 from the current endoscopic image D (Step S14).

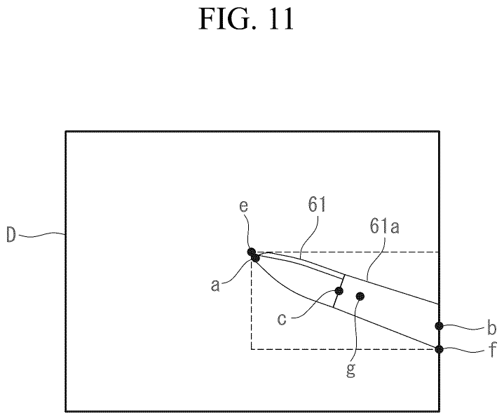

The position of each of the treatment tools 61 and 62 is, for example, the coordinates of at least one feature point in a region of the corresponding one of the treatment tools 61 and 62 recognized by the recognition unit 42. An example of the feature point is a distal end a or a proximal end b of the treatment tool 61, a distal end c of a shaft 61a thereof, the center of gravity g of the treatment tool 61, or a corner e or a corner f of a rectangle surrounding the treatment-tool region, as shown in FIG. 11.

The determination unit 44 obtains, from the storage unit 45, a past orientation and a past position (second position) of the follow target (Step S15). The past orientation and position obtained at this time are the orientation and the position of the follow target in a past endoscopic image D, which are the orientation and the position stored in the storage unit 45 in Step S19 the predetermined time before the current orientation and the current position, as will be described later.

Next, the determination unit 44 determines whether or not the endoscope 2 follows the movements of the treatment tools 61 and 62, on the basis of the current orientations and the current positions of the treatment tools 61 and 62, respectively.

Specifically, the determination unit 44 compares the current orientation and the current position (first position) of the treatment tool 61 with the past orientation and the past position of the follow target 61, respectively. In the case where the current orientation is the same or substantially the same as the past orientation of the follow target, and the current position is the same or substantially the same as the past position of the follow target, the determination unit 44 determines that the endoscope 2 follows the movement of the treatment tool 61, i.e., that the treatment tool 61 is the follow target (Step S16). On the other hand, in the case where the current orientation is neither the same nor substantially the same as the past orientation of the follow target, and/or the current position is neither the same nor substantially the same as the past position of the follow target, the determination unit 44 determines that the endoscope 2 does not follow the movement of the treatment tool 61 (Step S16).

The determination unit 44 performs similar determination for the other treatment tool 62.

For example, the determination unit 44 calculates a deviation d′ from Equation (2) and determines that the treatment tool of which the deviation d′ is equal to or smaller than the predetermined threshold and is the smallest is the follow target. The deviation d′ is the sum of a deviation of the current position p^{t} from the past position p^{t-i} and a deviation of the current orientation q^{t} from the past orientation q^{t-i}.

$${d}^{′} = ‖ {p}^{t} - {p}^{t - i} ‖ + ‖ {q}^{t} - {q}^{t - i} ‖ \qquad ( 2 )$$

The storage unit 45 stores the current orientation and the current position of the treatment tool 61 when the treatment tool 61 is determined to be the follow target by the determination unit 44 (Step S19).

According to this embodiment, the follow target is determined on the basis of the current orientations and the current positions of the treatment tools 61 and 62. Accordingly, it is possible to further improve the recognition accuracy of the follow target.

In this embodiment, after it is determined that the follow target is not present, the control device 4 may also automatically place the follow target. In this case, after Step S7, the control device 4 repeats Steps S2 to S7 until the treatment tool of which the deviation d′ is equal to or smaller than the threshold is found, and sets, to the follow target, the treatment tool of which the deviation d′ is equal to or smaller than the threshold, whereby placing the follow target. Accordingly, the treatment tool, the current orientation and current position of which are the same or substantially the same as the orientation and position of the follow target immediately before it is determined that the follow target is not present, is placed as the follow target.

For example, the orientation and the position of the follow target do not change between before and after the follow target disappears, unless the endoscope is not moved during the replacement of the treatment tool. Therefore, even after the control device 4 once lost sight of the follow target, it is possible to recognize the follow target again on the basis of the orientations and the positions of the treatment tools.

### Third Embodiment

Next, an endoscope system according to a third embodiment of the present disclosure will be described below.

An endoscope system 1 of this embodiment differs from the first embodiment in that whether or not the endoscope 2 follows the treatment tools 61 and 62 is determined on the basis of pivot points P1 and P2 of the treatment tools 61 and 62 in addition to the current orientations of the treatment tools 61 and 62 and the past orientation of the follow target. In this embodiment, configurations different from those in the first and second embodiments will be described below, identical reference signs are assigned to configurations common to those in the first and second embodiments, and a description thereof will be omitted.

As in the first embodiment, the endoscope system 1 of this embodiment includes the endoscope 2, the movement device 3, the control device 4, and the display device 5.

In this embodiment, the positions of the pivot points P1 and P2 of all the treatment tools 61 and 62 are set in the control device 4 and stored in the storage unit 45. For example, before the endoscope 2 is inserted into the body, a setting button (not shown) is pressed in a state in which the distal end of the endoscope 2 is arranged at the pivot point P1, whereby the teaching work for teaching the position of the pivot point P1 to the control device 4 is executed by an arbitrary worker such as an operator. The control device 4 calculates the position of the distal end of the endoscope 2 in a world coordinate system from the angles of the joints 3b of the robot arm 3a when the setting button is pressed, and sets the calculated position of the distal end of the endoscope 2 to the position of the pivot point P1. The world coordinate system is a coordinate system fixed to the proximal end of the robot arm 3a. Next, the control device 4 projects the pivot points P1 and P2 onto the image plane of the endoscopic image D through coordinate transformation and calculates the positions of the pivot points P1 and P2 in an image coordinate system of the endoscopic image D.

The determination unit 44 determines the follow target by determining whether or not the endoscope 2 follows the movements of the treatment tools 61 and 62, on the basis of the current orientations of the treatment tools 61 and 62 and the past orientation of the follow target, as in the first embodiment. Then, for example, in the case where it is determined that the deviations d of all the treatment tools 61 and 62 are larger than the threshold, i.e., that the treatment tool corresponding to the follow target is not present in the endoscopic image D, the determination unit 44 then obtains the pivot points P1 and P2 from the storage unit 45 and determines whether or not the endoscope 2 follows the treatment tools 61 and 62, on the basis of the positions of the pivot points (first pivot point) P1 and P2 of the treatment tools 61 and 62.

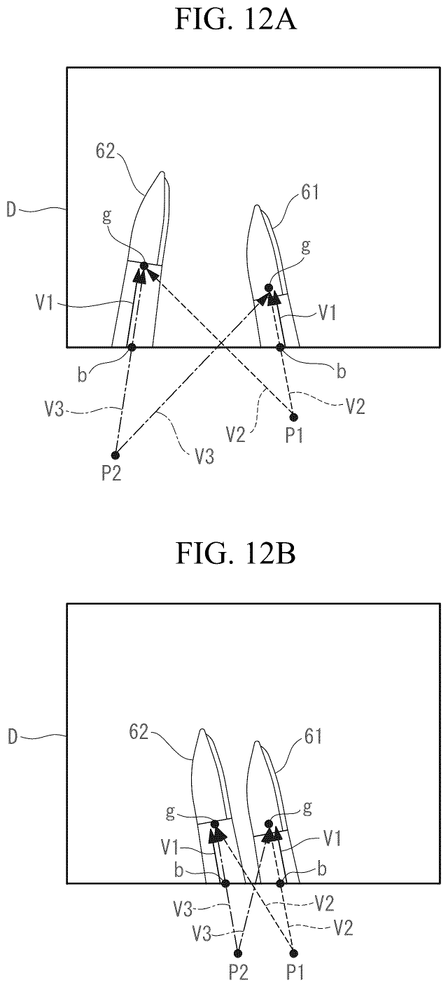

Specifically, as shown in FIGS. 12A and 12B, the determination unit 44 calculates, for each of the treatment tools 61 and 62 in the current endoscopic image D, a first vector V1 corresponding to the orientation of the treatment tool and a second vector V2 connecting the treatment tool and the pivot point (second pivot point) P1 of the follow target. The first vector V1 is, for example, a vector connecting the proximal end b and the center of gravity g of each of the treatment tools 61 and 62 in the endoscopic image D. The second vector V2 is, for example, a vector connecting the pivot point P1 of the follow target and the center of gravity g of each of the treatment tools 61 and 62, on the image plane of the endoscopic image D.

In the case of the follow-target treatment tool 61, the first vector V1 is parallel to the second vector V2. In the case of the treatment tool 62, which is not the follow target, the first vector V1 forms an angle with respect to the second vector V2.

The determination unit 44 calculates, for the treatment tool 61, the angle θ between the first vector V1 and the second vector V2. The determination unit 44 determines that the endoscope 2 follows the movement of the treatment tool 61, in the case where the angle θ is equal to or smaller than a predetermined threshold, and determines that the endoscope 2 does not follow the movement of the treatment tool 61, in the case where the angle θ is larger than the predetermined threshold.

Similarly, the determination unit 44 calculates, for the other treatment tool 62, the angle θ between the first vector V1 and the second vector V2. The determination unit 44 determines that the endoscope 2 follows the movement of the treatment tool 62, in the case where the angle θ is equal to or smaller than the predetermined threshold, and determines that the endoscope 2 does not follow the movement of the treatment tool 62, in the case where the angle θ is larger than the predetermined threshold.

The treatment tool 61, of which the angle θ is equal to or smaller than the predetermined threshold, is a candidate for the follow target. Therefore, as the result of the determination, the determination unit 44 extracts the candidate for the follow target from among the plurality of treatment tools 61 and 62.

If the treatment tool of which the angle θ is equal to or smaller than the predetermined threshold is not present, the determination unit 44 determines that the treatment tool corresponding to the follow target is not present in the endoscopic image D and notifies the determination result to the target setting unit 41.

Next, the determination unit 44 calculates, for each of the treatment tools 61 and 62 in the current endoscopic image D, a third vector V3 connecting the treatment tool and the pivot point P2 of the treatment tool 62, which is not the follow target, and calculates the angle θ′ between the first vector V1 and the third vector V3. Then, among the candidate(s), the determination unit 44 excludes the treatment tool of which the angle θ′ is equal to or smaller than the threshold from the candidate(s) and determines the remaining candidate to be the follow target.

As shown in FIG. 12A, in the case where the pivot point P1 of the follow target 61 is away from the pivot point P2 of the other treatment tool 62, the angle θ of the other treatment tool 62 is larger than the predetermined threshold, and thus the other treatment tool 62 is not misrecognized as the follow target. On the other hand, as shown in FIG. 12B, in the case where the pivot point P2 of the other treatment tool 62 exists near the pivot point P1 of the follow target 61, the angle θ of the other treatment tool 62 is also equal to or smaller than the predetermined threshold, whereby there is a possibility that the other treatment tool 62 is misrecognized as the follow target.

As described above, the treatment tool of which the angle θ and the angle θ′ are both equal to or smaller than the threshold is excluded from the candidate(s) for the follow target, thereby making it possible to prevent misrecognition of the follow target.

Next, the operation of the endoscope system 1 will be described below.

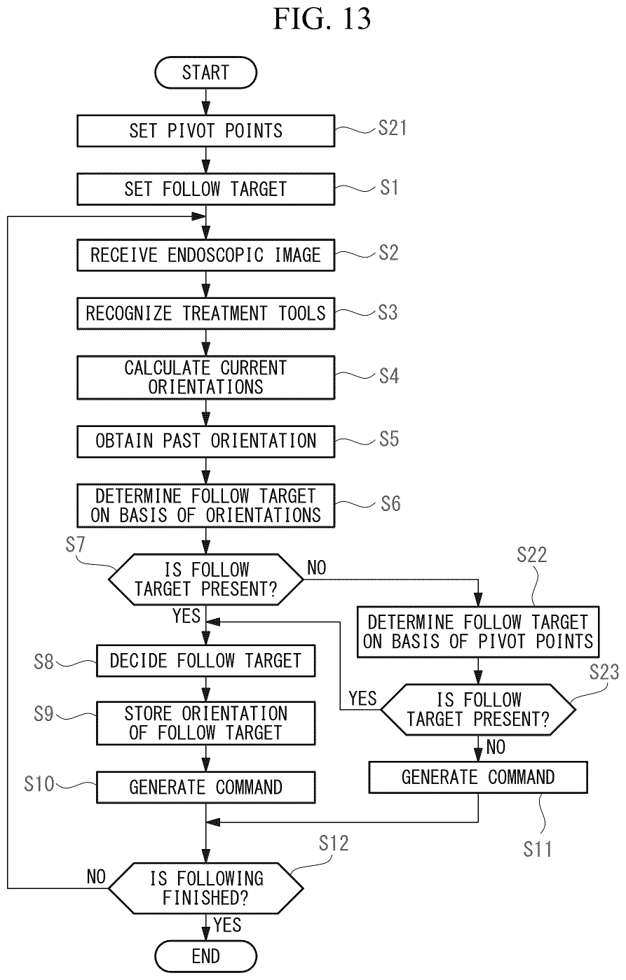

As shown in FIG. 13, the positions of the pivot points P1 and P2 of the treatment tools 61 and 62 in the image coordinate system are set in the control device 4 (Step S21), and then Steps S1 to S7 are executed as in the first embodiment. Then, in the case where it is determined that the treatment tool corresponding to the follow target is not present (NO in Step S7), the determination unit 44 obtains the pivot points P1 and P2 from the storage unit 45 and determines the follow target on the basis of the pivot points P1 and P2 (Step S22).

Specifically, the determination unit 44 calculates the first vector V1 corresponding to the current orientation of the treatment tool 61 and the second vector V2 from the pivot point P1 to the treatment tool 61, calculates the angle θ between the two vectors V1 and V2, and, in the case where the angle θ is equal to or smaller than the threshold, selects the treatment tool 61 as a candidate for the follow target. Similarly, the determination unit 44 calculates the angle θ for the other treatment tool 62 as well and, in the case where the angle θ is equal to or smaller than the threshold, selects the treatment tool 62 as a candidate for the follow target. Next, the determination unit 44 calculates, for each of the treatment tools selected as the candidates, the third vector V3 connecting the treatment tool and the pivot point P2 of the treatment tool 62, which is not the follow target, and calculates the angle θ′ between the first vector V1 and the third vector V3. Then, the determination unit 44 excludes, from the candidates, the treatment tool of which the angle θ′ is equal to or smaller than the threshold, determines the remaining candidate to be the follow target (YES in Step S23), and decides that the remaining candidate is the follow target (Step S8).

In the case where it is determined that the treatment tool corresponding to the follow target is not present in Step S23, the control device 4 repeats Steps S2 to S8, S22, and S23 until the follow target is found, thereby automatically placing the follow target.

In this way, according to this embodiment, in the case where the follow target cannot be determined on the basis of the current orientations of the treatment tools 61 and 62, the follow target is then determined on the basis of the pivot points P1 and P2. Accordingly, it is possible to prevent the control device 4 from losing sight of the follow target.

In this embodiment, the determination unit 44 may determine that the treatment tool of which the angle θ is equal to or smaller than the predetermined threshold and is the smallest is the follow target, without narrowing down the candidates on the basis of the angle θ′, in Step S22. In this case, only the position of the pivot point P1 of the follow target may be set in Step S21.

In this embodiment, although the determination unit 44 determines the follow target on the basis of the current orientations and the past orientation, instead of this, it is also possible to determine the follow target on the basis of the current orientations and the pivot points P1 and P2. Furthermore, the determination unit 44 may narrow down the candidates for the follow target on the basis of the pivot points P1 and P2.

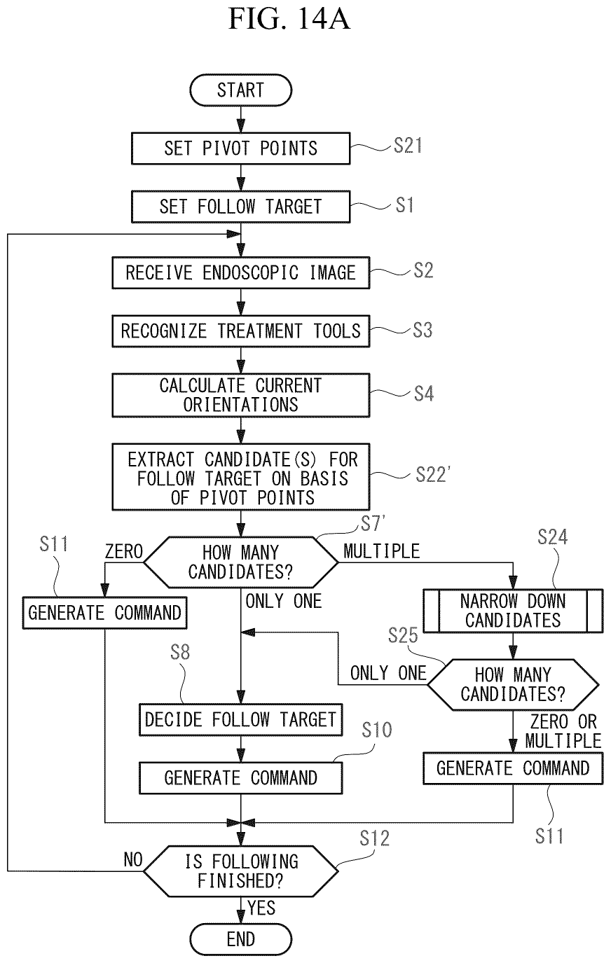

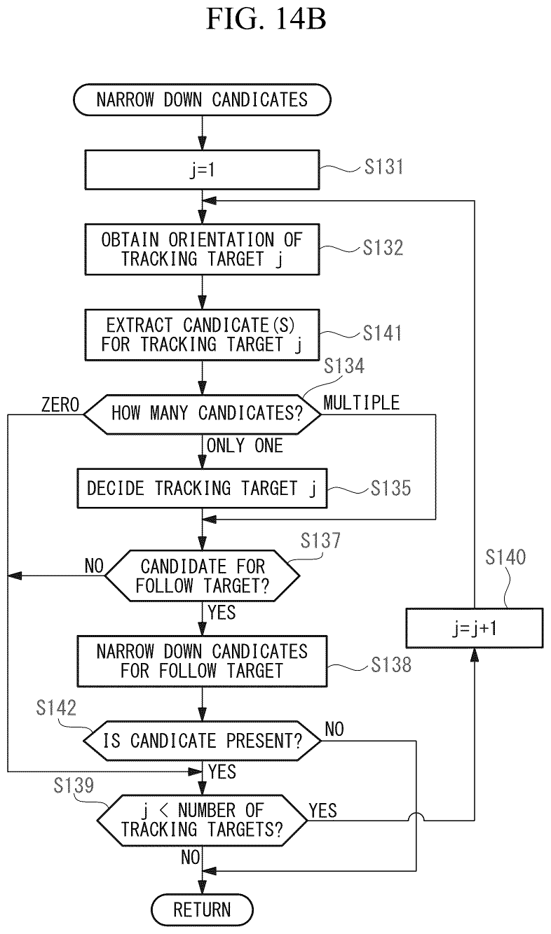

That is, as shown in FIG. 14A, in Step S22′, the determination unit 44 selects the treatment tool of which the angle θ is equal to or smaller than the predetermined threshold, as a candidate for the follow target, as in the Step S22.

In the case where the number of selected candidates for the follow target is multiple (“multiple” in Step S7′), the determination unit 44 narrows down the candidates (Step S24). Specifically, as shown in FIG. 14B, in Step S24, the determination unit 44 selects, from the candidates, one treatment tool as a tracking target j (Step S131) and extracts a candidate(s) for the tracking target j on the basis of the orientation and the pivot point of the tracking target j (Step S141). Specifically, the determination unit 44 calculates a first vector corresponding to the orientation of the tracking target j and a second vector connecting the pivot point of the tracking target j and each of the treatment tools, and selects the treatment tool of which the angle θ between the first vector and the second vector is equal to or smaller than the predetermined threshold, as the candidate for the tracking target j.

In the case where the number of candidates is one or multiple, the determination unit 44 determines whether or not the candidate(s) for the tracking target j is included in the candidates for the follow target (Step S137). In the case where the candidate(s) for the tracking target j is included in the candidates for the follow target, the determination unit 44 deletes the candidate(s) for the tracking target j from the candidates for the follow target (Step S138).

The determination unit 44 sequentially selects, as the tracking target j, one treatment tool that is the candidate for the follow target and executes processing of Steps S132 to S138 (Step S139, S140). Accordingly, the candidates for the follow target are narrowed down.

In the case where the number of candidates for the follow target becomes zero before all the treatment tools that are the candidates for the follow target are selected as the tracking target j (NO in Step S142), the determination unit 44 may finish narrowing down the candidates at this point.

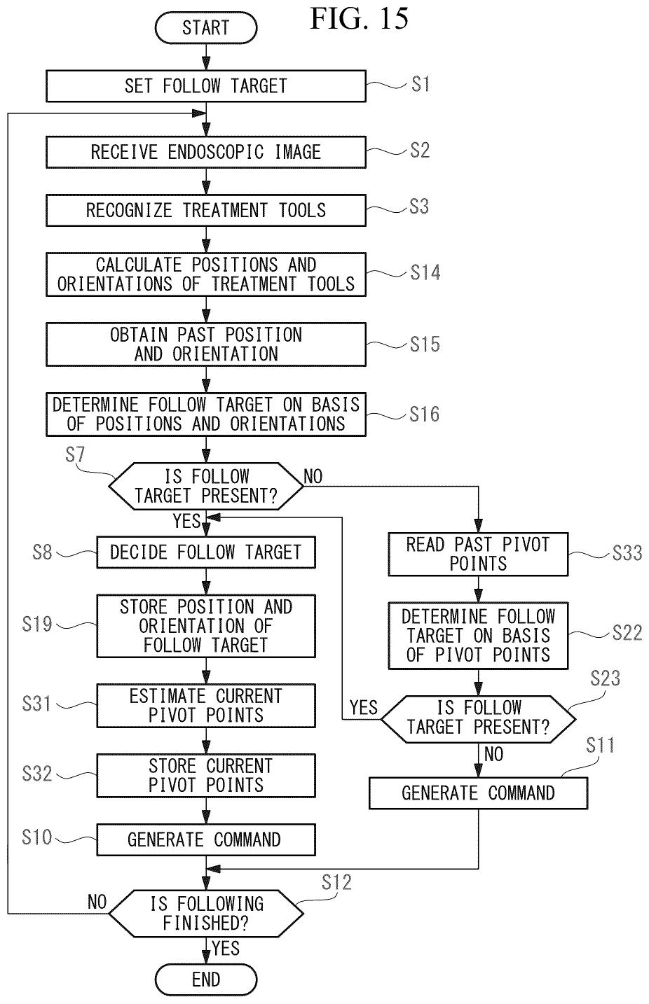

In this embodiment, the control device 4 may determine whether or not the endoscope 2 follows the movements of the treatment tools 61 and 62, on the basis of the current orientations, the current positions, and the pivot points P1 and P2 of the treatment tools 61 and 62 and the past orientation and the pivot point P1 of the follow target. That is, as shown in FIG. 15, the control device 4 may execute, instead of Steps S4 to S6, Steps S14 to S16 of the second embodiment.

Furthermore, in this embodiment, although the positions of the pivot points P1 and P2 are set prior to tracking of the follow target, instead of this, as shown in FIG. 15, the determination unit 44 may estimate the positions of the pivot points P1 and P2 from the current endoscopic image D.

In the modification shown in FIG. 15, after the follow target is decided (Step S8), the current positions of the pivot points P1 and P2 of the treatment tools 61 and 62 are estimated by using the current endoscopic image D (Step S31), and the positions of the pivot points P1 and P2 are stored in the storage unit 45 (Step S32). Then, after it is determined that the treatment tool corresponding to the follow target is not present in Step S7, the past positions of the pivot points P1 and P2 that have already been stored in the storage unit 45 are obtained (Step S33), and the follow target is determined by using the past positions of the pivot points P1 and P2 (Step S22).

According to this modification, the work of setting the positions of the pivot points in Step S21 can be eliminated.

In Step S31, the determination unit 44 estimates the position of the pivot point P1 of the treatment tool 61 in the current endoscopic image D by using the position of the treatment tool 61, depth information of the treatment tool 61, and the position and the orientation of the endoscope 2 in the endoscopic image D. Similarly, the determination unit 44 also estimates the pivot point P2 of the other treatment tool 62 in the current endoscopic image D.

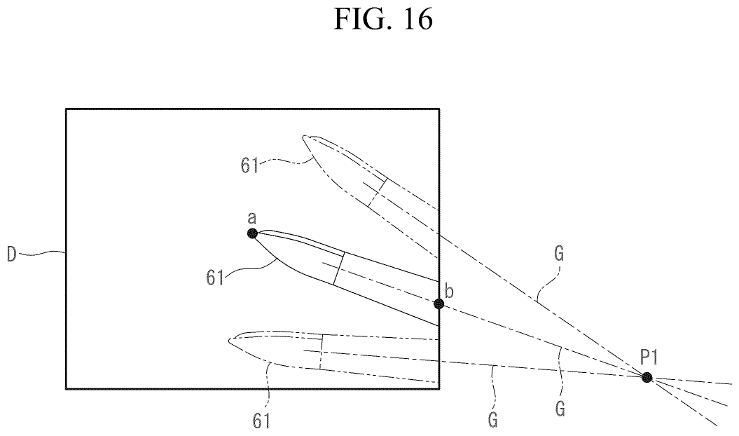

Specifically, as shown in FIG. 16, the determination unit 44 calculates 2D positions of the distal end a and the proximal end b of the treatment tool 61 in the image coordinate system, from the current endoscopic image D. Furthermore, the determination unit 44 obtains the positions of the distal end a and the proximal end b of the treatment tool 61 in the depth direction by a 3D measurement means such as a 3D camera. Then, the determination unit 44 converts the 2D positions of the distal end a and the proximal end b in the image coordinate system into the 3D positions of the distal end and the proximal end in a camera coordinate system, by using the positions of the distal end a and the proximal end b in the depth direction. Next, the determination unit 44 calculates the position and the orientation of the distal end of the endoscope 2 in the world coordinate system, from the angles of the joints 3b of the robot arm 3a, and converts the 3D positions of the distal end a and the proximal end b in the camera coordinate system into the positions of the distal end a and the proximal end b in the world coordinate system, by using the position and the orientation of the distal end of the endoscope 2 in the world coordinate system. Then, the determination unit 44 calculates the longitudinal axis G, of the treatment tool 61, connecting the distal end a and the proximal end b in the world coordinate system, obtains a past longitudinal axis G from the storage unit 45, and calculates the point of intersection of the two longitudinal axes G as the pivot point P1. The calculated position of the pivot point P1 is stored in the storage unit 45.

### Fourth Embodiment

Next, an endoscope system according to a fourth embodiment of the present disclosure will be described below.

An endoscope system 1 of this embodiment differs from the first embodiment in that the determination of the follow target based on the deviations d of the orientations of the treatment tools 61 and 62 is not performed, and whether or not the endoscope 2 follows the movements of the treatment tools 61 and 62 is determined on the basis of the current orientations and the pivot points P1 and P2 of the treatment tools 61 and 62. In this embodiment, configurations different from those in the first to third embodiments will be described below, identical reference signs are assigned to configurations common to those in the first to third embodiments, and a description thereof will be omitted.

As in the first embodiment, the endoscope system 1 of this embodiment includes the endoscope 2, the movement device 3, the control device 4, and the display device 5.

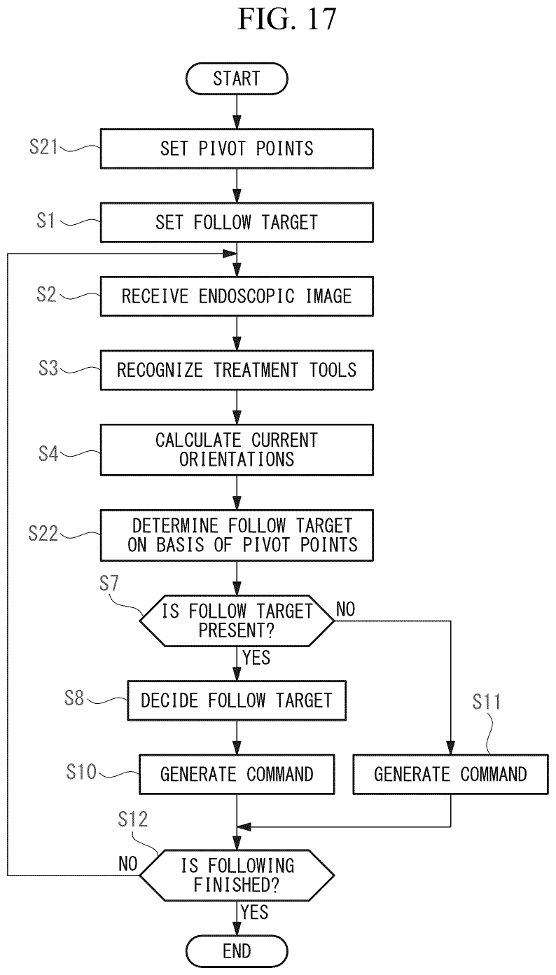

As shown in FIG. 17, after Step S4, the determination unit 44 determines the follow target on the basis of the positions of the pivot points P1 and P2 (Step S22). Step S22 is as described in the third embodiment.

In this way, according to this embodiment, the first vectors V1, which correspond to the current orientations of the treatment tools 61 and 62, and the second vectors V2, which connect the pivot point P1 of the follow target and the treatment tools 61 and 62, are calculated, and whether or not each of the treatment tools 61 and 62 is the follow target is determined on the basis of the angle θ between the corresponding vectors V1 and V2. Usually, the orientation and the pivot point P1 of the follow-target treatment tool 61 are not rapidly changed. Furthermore, the orientations of the plurality of treatment tools 61 and 62 in the endoscopic image D are different from each other. Therefore, whether or not each of the treatment tools 61 and 62 is the follow target can be accurately determined on the basis of the angle θ.

In this embodiment, in Step S22, it is also possible to determine that the treatment tool Of Which the angle θ is equal to or smaller than the predetermined threshold and is the smallest is the follow target, without narrowing down the candidates on the basis of the angle θ′ in Step S22.

In the above-described embodiments, the orientations and the positions calculated by the calculation unit 43 may be superimposed on the endoscopic image D.

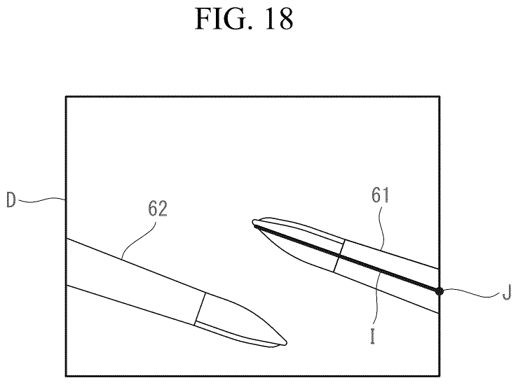

For example, as shown in FIG. 18, a line I indicating the longitudinal axis of the treatment tool 61, which serves as the orientation of the follow-target treatment tool 61, and a dot J indicating the proximal end b, which serves as the position of the treatment tool 61, may be superimposed on the follow target 61.

According to this configuration, in the case where the follow-target treatment tool 61 disappears from the endoscopic image D, the previous orientation I and the previous position J of the treatment tool 61 are kept being displayed, thereby making it possible to easily determine the follow target on the basis of the orientation I and the position J when a follow target is placed.

### REFERENCE SIGNS LIST

1 endoscope system
2 endoscope
3 movement device
4 control device
5 display device
6, 63 treatment tool
61 treatment tool (first treatment tool, second treatment tool)
62 treatment tool (third treatment tool)
P1 pivot point (first pivot point, second pivot point)
P2 pivot point

1. An endoscope system comprising:
an endoscope;
a robot arm that is configured to move the endoscope; and
a controller that is configured to control the endoscope and the robot arm,
wherein the controller is configured to:
receive an image acquired by the endoscope,
calculate a first orientation of a first treatment tool in a first image, which is the image received at a given point in time,
calculate a second orientation of a second treatment tool in a second image, which is the image received a predetermined time before the first image,
calculate a difference in orientation between the first orientation and the second orientation,
determine whether or not the endoscope follows movement of the first treatment tool by comparing the difference in orientation with a threshold value, and
in response to determining that the endoscope follows the movement of the first treatment tool, generate a command for the robot arm for making the endoscope follow the first treatment tool.

2. The endoscope system according to claim 1, wherein the controller is further configured to, in response to determining that the endoscope does not follow the movement of the first treatment tool, generate a command for the robot arm for making the endoscope stop following the first treatment tool.

3. The endoscope system according to claim 1, wherein the given point in time is a present time.

4. The endoscope system according to claim 1, wherein the second treatment tool is the first treatment tool.

5. The endoscope system according to claim 1, wherein the controller is further configured to:
calculate a first position of the first treatment tool,
calculate a second position of the second treatment tool,
calculate a difference in position between the first position and the second position, and
determine whether or not the endoscope follows the movement of the first treatment tool by comparing the difference in orientation with the threshold value and the difference in position with a further threshold value.

6. The endoscope system according to claim 5, wherein the second treatment tool is the first treatment tool.

7. The endoscope system according to claim 1, wherein the first orientation is an angle formed between a reference axis in the image and the first treatment tool.

8. The endoscope system according to claim 1, wherein the first orientation is a vector in a longitudinal direction of the first treatment tool in the image.

9. An endoscope system comprising:
an endoscope;
a robot arm that is configured to move the endoscope; and
a controller that is configured to control the endoscope and the robot arm,
wherein the controller is configured to:
receive an image acquired by the endoscope,
calculate an orientation of a treatment tool in an image,
obtain a position of a pivot point of the treatment tool,
calculate a position of a proximal end of the treatment tool,
calculate a first vector parallel to a first line passing through the position of the proximal end of the treatment tool and a given position on the treatment tool,
calculate a second vector parallel to a second line passing through the position of the pivot point and the given position,
move the endoscope to follow the movement of the treatment tool in a case in which an angle between the first vector and the second vector is equal to or less than a predetermined threshold value, and
in response to determining that the endoscope follows the movement of the treatment tool, generate a command for the robot arm for making the endoscope follow the treatment tool.

10. A method for controlling an endoscope system, the method comprising:
calculate a first orientation of a first treatment tool in a first image received from an endoscope at a given point in time,
calculate a second orientation of a second treatment tool in a second image received from the endoscope a predetermined time before the first image,
calculate a difference in orientation between the first orientation and the second orientation,
determine whether or not the endoscope follows movement of the first treatment tool by comparing the difference in orientation with a threshold value, and
in response to determining that the endoscope follows the movement of the first treatment tool, generate a command for a robot arm for making the endoscope follow the first treatment tool.
